(19) United States
(12) Patent Application Publication
(10) Pub. No.: US 2023/0024537 A1
(43) Pub. Date: Jan. 26, 2023

(54) Oral Delivery Compositions for Obesity Management

(71) Applicant: BOARD OF REGENTS, THE UNIVERSITY OF TEXAS SYSTEM, Austin, TX (US)

(72) Inventors: Md Nurunnabi, El Paso, TX (US); Humayra Afrin, El Paso, TX (US); Md Nurul Huda, El Paso, TX (US); Christiancel Joseph J. Salazar, El Paso, TX (US)

(21) Appl. No.: 17/871,953

(22) Filed: Jul. 24, 2022

Related U.S. Application Data
(60) Provisional application No. 63/225,477, filed on Jul. 24, 2021.

(51) Int. Cl. C07K 16/26; A61K 9/51; A61K 9/00; A61P 3/04

(52) U.S. Cl. CPC C07K 16/26; A61K 9/5115; A61K 9/0056; A61P 3/04; A61K 2039/505

(57) ABSTRACT

Certain embodiments are directed to compositions and related methods for oral delivery of compositions for effective administration of leptin pathway modulating agents (e.g., leptin, anti-leptin antibodies, anti-leptin receptor antibodies and the like), the composition including an ionic liquid (e.g., CAGE) or a beta-glucan composition and a leptin pathway modulator for reducing or maintaining body weight. In certain aspects the leptin pathway modulator is leptin or an anti-leptin antibody. An ionic liquid (IL) is a salt in the liquid state.

### PRIORITY

This application is a US Non-provisional application claiming priority to U.S. Provisional Application 63/225,477 filed Jul. 24, 2021, which is incorporated herein by reference in its entirety.

### FIELD OF THE INVENTION

Embodiments of the invention are directed generally to the field of medicine and pharmaceuticals. Particular embodiments of the invention are directed to oral delivery compositions for the treatment of obesity.

### BACKGROUND

The passing of foods and other important metabolically important compounds through the stomach and liver is known as the first pass. This first pass greatly reduces micronutrients, ergogenic supplements, pharmaceuticals, and other metabolically active compounds (“infusing compounds”) systematically available for absorption. Oral delivery of gastrointestinal labile agents is problematic.

Obesity is a chronic disease that is prevalent in modern society and is associated not only with a social stigma, but also with decreased life span and numerous medical problems—obesity is a primary risk factor for type II diabetes mellitus and is a strong risk factor for cardiovascular disease and cancer as well. Much of this obesity-induced pathology can be attributed to the strong association with dyslipidemia, hypertension, and insulin resistance (IR). Many studies have demonstrated that reduction in obesity by diet and exercise reduces these risk factors dramatically. Unfortunately, these treatments are largely unsuccessful with a failure rate reaching 95%. This failure may be due to the fact that the condition is strongly associated with genetically inherited factors that contribute to increased appetite, preference for highly caloric foods, reduced physical activity, and increased lipogenic metabolism. This indicates that people inheriting these genetic traits are prone to becoming obese regardless of their efforts to combat the condition.

One in every 3 Americans is living with obesity, a precondition that accompanies several diseases, including hypertension, diabetes, asthma, stroke, chronic back pain, and congestive heart failure. Obesity is considered a strong factor in many chronic diseases, including heart disease, diabetes, and cancer. Along with genetics, consumption of excessive fat and cholesterol-rich food are the primary causes of obesity.

Existing therapies for obesity include standard diets and exercise, very low calorie diets, behavioral therapy, pharmacotherapy involving appetite suppressants, thermogenic drugs, and food absorption inhibitors, mechanical devices such as jaw wiring, waist cords, balloons, and surgery (Jung and Chong. Clinical Endocrinology. 1991, 35:11-20; Bray. Am J Clin Nutr. 1992, 55:538S-544S). Because dieting and exercise produce only modest results, researchers have searched for a compound(s) to accelerate the loss of body fat. However, of the drugs currently allowed for use over a long period of time, sibutramine, an appetite depressant, and orlistat, a lipase inhibitor, have side effects including headaches, polydipsia (serious thirst), insomnia, constipation, hypertension, and increased pulse rate and fecal incontinence, frequent or urgent bowel movements, steatorrhea, and a reduction in the absorption of fat-soluble vitamins, respectively.

There remains a need for additional compositions and methods for the treatment of obesity.

### SUMMARY

One solution to the problems described above is to provide additional anti-obesity compositions that can be used to modulate the physiology of a subject and results in weight loss. Certain embodiments are directed to such a composition and related methods, such as an oral composition for effective for the treatment of obesity and/or the administration of a therapeutic agent (anti-obesity agent) for the treatment of obesity. Such therapeutic agents includes but is not limited to leptin pathway modulating agents (e.g., leptin, anti-leptin antibodies, anti-leptin receptor antibodies and the like), metal nanoparticles, and/or DHA (docosahexaenoic acid) and/or EPA (eicosapentaenoic acid). In certain aspects these therapeutic agents are administered in combination with or as a composite with an ionic liquid (e.g., CAGE) or a beta-glucan composition. The compositions and/or composites of the invention can be used for reducing or maintaining body weight. In certain aspects the leptin pathway modulator is leptin or an anti-leptin antibody. In other aspects the therapeutic is DHA, EPA, or an analog thereof. In certain aspects a metal nanoparticle or metal nanoparticle complex is incorporated into an ionic liquid forming an ionic liquid (IL) composite (IL-MNP). In certain aspects the ionic liquid composite includes a metal nanoparticle, DHA or EPA, or a metal nanoparticle and DHA or EPA.

An ionic liquid (IL) is a salt in the liquid state. Choline and geranate can both be used for formulating CAGE ionic liquid. In some embodiments, the ionic liquid comprises the choline cation and geranic acid anion in a molar ratio of 0.5:1, 1:1, 1:2, 1:3, or 1:4 of choline cation to geranic acid anion. In particular aspects, the choline cation and geranic acid anion are present in a molar ratio that is or is about 1:2 of choline cation to geranic acid anion. In some embodiments, the ionic liquid comprises the choline cation and geranic acid anion in a molar ratio in a range of 1:1 to 1:4 of choline cation to geranic acid anion. In some embodiments, the ionic liquid comprises the choline cation and geranic acid anion in a molar ratio of 0.5:1, 1:1, 1:2, 1:3, or 1:4 of choline cation to geranic acid anion. The choline cation can be derived from choline bicarbonate or the like.

A therapeutic agent can be added to the ionic liquid. In certain aspects the therapeutic agent is an anti-obesity therapeutic. In other aspects the anti-obesity therapeutic modulate the leptin pathway. The leptin pathway modulator can be a leptin protein or an anti-leptin antibody. A protein component, e.g., an anti-leptin antibody or leptin protein, can be added at a concentration of 10, 15, 20, 25, 30, 35, to 40% (w/v). The ionic liquid is a solubilizer of many hydrophobic and hydrophilic drugs on top of oral enhancer. The ionic liquid facilitates to dissolve the antibody and protein, and maintains stability for 2 to 3 months at room temperature and 6 to 12 months at 4° C.

The ionic liquid composition may be associated with or packaged in a delivery system such as a coating, capsule, or a co-crystal. The ionic liquid composition or composite can be encapsulated in a shell or capsule. A capsule or shell can be filled with the leptin antibody or protein, metal nanoparticles, DHA (docosahexaenoic acid) and/or EPA (eicosapentaenoic acid), or any combination thereof. The ionic liquid can be administered in a volume of 10, 20, 30, 40 to 50 μL/capsule. The ionic liquid filled capsule can be stored at 4° C. if not immediately used.

In some embodiments, the ionic liquid comprises a concentration of about 0.1% to 99% of the IL composition, and the pharmaceutically acceptable solvent comprises a concentration of about 1% to about 99.9% of the composition. Both choline and geranate acid are recognized by the Food and Drug Administration as generally regarded as safe (GRAS) ingredients. Choline, an important constituent of lecithin (fat) that is present in both plants and animals and required for various physiological functions. Beta-glucan is a non-starch polysaccharide, prebiotic fiber formed primarily by β-1,3 and 1,6 glycosidic bonds. Beta-glucans, particularly those found in cereal grains, have been shown in research to help lower cholesterol levels, beneficiary role in obesity, metabolic disorders, and other chronic non-communicable diseases. They also play a significant role in promoting a healthy microbiome via stimulating growth of beneficial bacteria species, mitigating pathogenic ones, and modulating inflammation to optimize the gut environment. They are now ubiquitous in health food stores across the country, mainly for their immunomodulatory properties and cancer-protective effects. They are present in a variety of other food products, such as oats, barley, seaweeds, and yeast, as well as in dietary supplements.

Leptin protein stability was assessed for oral formulations. Leptin protein concentration is 0.1, 0.25, 0.5, 0.75, 1, 1.25, 1.5, 1.75, to 2 mg/ml, including all values and ranges there between. Leptin can be added to BG and CAGE compositions in a ration of 1:1, 1:2, 1:3, 1:4, 1:5, 1:6, 1:7, 1:8, 1:9, to 1:10 molar volume (leptin:BG or CAGE). For both CAGE and BG compositions the protein and formulation were incubated for 30 min, 1 h, 2 h and 4 h time point. After the incubation period, proteins were denatured and equal volume loaded in each well of a polyacrylamide gel. A 12% polyacrylamide gel and 8 wells were used in one example to run samples with protein ladder and lysis buffer.

Beta-glucan compositions can be used to delivery other nutrients or therapeutic molecule via oral delivery. The beta glucan gel facilitates oral delivery of various nutraceuticals including iron sulfate, omega 3 fatty acid, vitamin B12 and DL-Thioctic acid. The regular absorption rate for iron sulfate is ˜1% but Beta glucan enhances absorption by 60% (60 folds) when given orally. Enhanced absorption of iron sulfate by beta glucan resulted in mitigation of constipation problem, a very common problem associated with oral iron sulfate delivery. Utilization of this beta glucan gel mediated iron sulfate oral delivery has potential to reduce the dose by 50-70%. Beta glucan mediated oral delivery of omega 3 fatty acid (Docosahexaenoic acid) resulted in enhancement of pharmacokinetics by 10 folds. Beta glucan associated oral delivery of vitamin B12 delivery resulted in 15-20 folds higher pharmacokinetics. Beta glucan associated oral delivery of DL-thioctic acid resulted in increased pharmacokinetics by 10-20 folds compare to free DL-thioctic acid oral delivery, tested in rat.

The inventors have developed an oral antibody or protein delivery platform and investigated feasibility using an anti-leptin antibody. The oral delivery of anti-leptin antibody over 30 days resulted in reduction of 15-35% body weight reduction in a high fat diet induced rat model. The oral delivery vehicle facilitated enhancement of intestinal penetration of the antibody as well as protect the antibody from being damaged within the stomach in presence of acidic gastric juice. An oral dose of 5-10 μg antibody resulted in 15-35% body weight reduction compared to untreated high fat diet rat. An oral dose of 5-10 μg leptin protein resulted 10-20% body weight reduction compared to the untreated high fat diet rat. Both, leptin antibody and hormone treated rat shows significantly less fat content in their body. Treated groups show less cholesterol contents in their blood which is within the normal range in opposed to the untreated animal that are observed with high cholesterol content. Leptin antibody and protein treated rat were observed to consume 10-15% less food than the untreated rats. The orally administered anti leptin antibody results in neutralizing circulating leptin hormone which is a readout for therapeutic efficacy. The oral anti leptin antibody delivery shows potential of treating individual who are obese due to leptin hormone resistant. The oral leptin protein results in production anti leptin antibody.

Administration of the compositions can achieve sustained release or long-term delivery. By “sustained release or” “long term release” as used herein is meant that the delivery system administers a therapeutic amount of subject compounds for more than a day, preferably more than a week, and most preferable at least about 30 days to 60 days, or longer.

The terms “inhibiting,” “reducing,” or “prevention,” or any variation of these terms, when used in the claims or the specification includes any measurable decrease or complete inhibition to achieve a desired result.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Leptin Protein stability of both (A) β-Glucan (BG) and (B) CAGE oral delivery. 1=Protein Ladder, 2=Leptin protein, 3=BG-Leptin protein 4-hour incubation, 4=BG-Leptin protein 2-hour incubation, 5=BG-Leptin protein 1-hour incubation, 6=BG-Leptin protein 30-minute incubation, 7=CAGE-Leptin protein 30 minute incubation, 8=CAGE-Leptin protein 1-hour incubation, 9=CAGE-Leptin protein 2-hour incubation, 10=CAGE-Leptin protein 4-hour incubation, 11=CAGE-Leptin protein 8-hour incubation.

FIG. 2. Male rat body weight changes with treatment. Male with leptin protein treatment gain body weight lower than regular and high fat diet male control group

FIG. 3. Male Rat CAGE-Leptin Antibody treatment study. Four groups of male were getting high fat diet for 2 weeks and then we start administer with cage, cage-leptin ab and leptin ab treatments.

FIG. 4. Food consumption by male rat.

FIG. 5. Average food consumption by male rat.

FIG. 6. Female rat body weight changes with leptin protein treatments.

FIG. 7. CAGE and Leptin antibody treatment of rat.

FIG. 8. CAGE and Leptin antibody treatment study.

FIG. 9. Female rat food consumption.

FIG. 10. Female rat average food consumption.

FIG. 11. Fat accumulation in stomach and intestinal region.

FIG. 12. Serum analysis of leptin project: BG treatment.

FIG. 13. Serum analysis of leptin project: CAGE treatment.

FIG. 14. Schematic of the process for synthesizing magnet nanoparticle (MNP) conjugated ionic liquid (IL).

FIG. 15. Transmission Electron Microscopy of MNP and IL+MNP.

FIG. 16. Illustration of size and zeta potential of IL+MNP composites.

FIG. 17. Illustration of size distribution of IL+MNP composites.

FIG. 18. SPR analysis of IL+MNP composites.

FIG. 19. Changes in body weight and food uptake of rats administered IL+MNP composites.

FIG. 20. Effects of IL and IL+MNP composites on body weight and food uptake under regular diet conditions.

FIG. 21. Illustration of the mechanism of action related to IL and IL+MNP composites.

FIG. 22. Effects of IL and IL+MNP on serum biochemistry.

FIG. 23, Histology of major organs after 15 days of oral administration of IL and IL+MNP composites.

FIG. 24. Histology of the small and large intestines after 15 days of administration of IL and IL+MNP composites.

FIG. 25. Serum GLP1 receptor levels after administration of IL or IL+MNP composites.

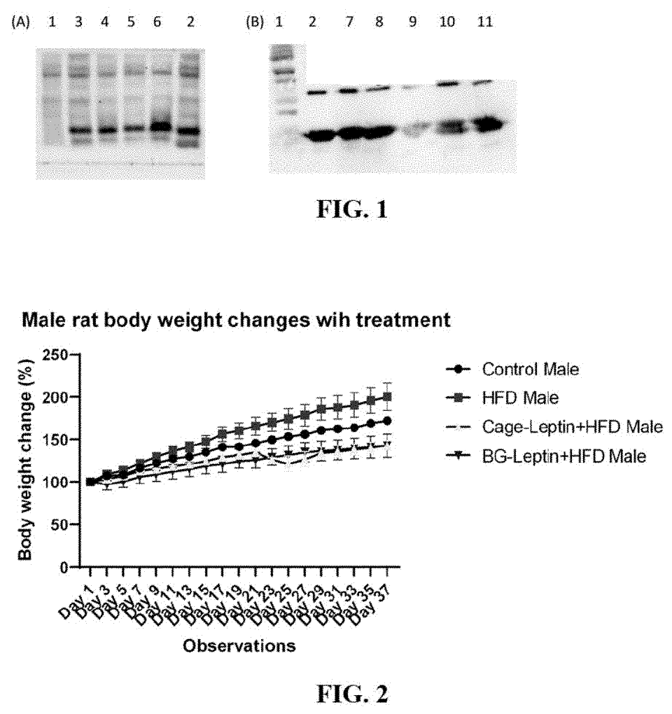

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Glucans are a heterogeneous group of glucose polymers found in the cell walls of plants, bacteria, fungi and protozoa. Glucans have a backbone chain and in some cases side chains which, depending of the origin of the glucan, comprise β(1,3), β(1,4) and/or β(1,6)-linked glucosyl units. Depending upon the source and method of isolation, beta-glucans have various degrees of branching and type of linkage in the backbone and side chains. The frequency and type of linkage in the side chains is highly relevant to the biological activity of the molecule. Glucans also differ highly in their molecular weight as well as in their tendency for chain aggregation which both are essential features for the efficacy profile of these molecules. Most beta-glucans of fungal and yeast origin are in their native state insoluble in water but can be made soluble either by acid hydrolysis or by derivatization introducing foreign groups like—phosphate, -sulphate, -amine, -carboxymethyl and so forth to the molecule.

Leptin is secreted primarily by fat cells and acts centrally, particularly in the hypothalamus, to reduce food intake and body weight. Classical JAK2 (Janus kinase-2)-STAT3 (signal transducer and activator of transcription-3) pathway, play an important role in mediating leptin signaling in the hypothalamus. Leptin action in the hypothalamus is mediated by an insulin-like signaling pathway involving stimulation of PI3K (phosphatidylinositol-3 kinase) and PDE3B (phosphodiesterase-3B), and reduction in cAMP levels. This means a PI3K-PDE3B-cAMP pathway interacting with the JAK2-STAT3 pathway constitutes a critical component of leptin signaling in the hypothalamus. Leptin is a protein hormone, that is primarily segregated by the fat cells (adipocytes). In 1994, leptin (Zhang et al. (1994) Nature 372, 425) was discovered as genetic product of the obesitas gene (ob gene) with a molecular weight of approx. 16 kDa, formed by 146 amino acids. It plays an important role in energy metabolism (Friedman et al., (1998) Nature 395, 763-770). Apart from its relevance for energy metabolism, its contribution to the modulation of immunocompetent cells (Lord et al., (1998) Nature 394, 6696) or haematopoietic cells (Sierra-Honigmann et al., (1998) Science 281, 1683-1686) has been described in the meantime as well as a permissive function in the induction of puberty (Quinton et al., (1999) J Clin Endocrinol Metab 84(7), 2336-41). Leptin has also been strongly associated with diabetes and chronic heart failure (CHF) (see E. M. El-Bindary and A. Z. Darwish, Volume 7, Nos 4/5, July-September 2001, 697-706).

In mice (ob/ob mouse), the lack of the leptin gene (ob/ob mouse) leads to massive overweight (adipositas). Due to the fact, that administration of leptin to ob/ob mice induces reduced food uptake and finally weight reduction, leptin was attributed some importance as an appetite suppressant. Anyhow, the situation is far more complex.

The effect of leptin is mediated via the leptin receptor (leptin-R) (Tartaglia et al., (1995) Cell 83(7), 1263-71), thus activating intracellular signal cascades. The leptin receptor pertains to the so-called class I of the cytokine receptor superfamily. In many receptors of this family, an extra-cellular portion of the leptin receptors circulates as leptin-binding protein in the blood, and this is also the case for the leptin receptor.

Defects of the leptin receptor were identified as a cause of overweight problems in human beings (Clement et al., (1998) Nature 392, 398-401). Conversely, patients suffering from anorexia nervosa (diminished appetite) seemingly have increased leptin levels in relation to their reduced fat mass. Therefore, determination of leptin concentration in blood or in serum samples is an important diagnostic tool for clarification of the underlying cause of eating disorders or extreme obesity.

Docosahexaenoic acid (DHA, 22:6) and eicosapentaenoic acid (EPA, 20:5) have been reported to improve metabolic disorders. 1% DHA and 1% EPA inhibit adipogenesis have been shown to down-regulating GPR120. It has also been shown that 4% DHA stimulates browning of WAT and improves insulin resistance and inflammatory infiltration by up-regulating PPARγ.

### I. IONIC LIQUID COMPOSITIONS

Described herein are compositions comprising an ionic liquid comprising a choline cation and a fatty acid anion. In some embodiments, the composition further comprises a pharmaceutically acceptable solvent. In some embodiments, the fatty acid is myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is geranic acid. In some embodiments, the ionic liquid is liquid at room temperature. In some embodiments, the ionic liquid is liquid below 100° C.

In some embodiments, the ionic liquid comprises a molar ratio of a choline cation to a fatty acid anion of 1:0.5 to 1:10. In some embodiments, the molar ratio of the choline cation to the fatty acid anion is about 1:0.5, 1:0.6, 1:0.7, 1:0.8, 1:0.9, 1:1.0; 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9. 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, 1:8.1, 1:8.2, 1:8.3, 1:8.4, 1:8.5, 1:8.6, 1:8.7, 1:8.8, 1:8.9, 1:9.0, 1:9.1, 1:9.2, 1:9.3, 1:9.4, 1:9.5, 1:9.6, 1:9.7, 1:9.8, 1:9.9, or about 1:10. In some embodiments, the molar ratio of the choline cation to the fatty acid anion is about 1:1.1, 1:1.2, 1:1.3, 1:1.4, 1:1.5, 1:1.6, 1:1.7, 1:1.8, 1:1.9, or 1:2.0.

The chemical structure of choline is:

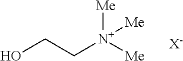

In some embodiments, term choline refers to the class of quaternary ammonium salts containing the N,N,N-trimethylethanolammonium cation. In some embodiments, the X— on the right of the structure of choline denotes a pharmaceutically acceptable anion. In some embodiments the X— is bicarbonate, carbonate, acetate, citrate, tartarate, bitartarate, lactate, chloride, bromide, or iodide. In some embodiments, the X— is bicarbonate. In some embodiments, the choline is an anti-inflammatory agent.

In some embodiments, choline is in the form of a pharmaceutically acceptable salt. The type of pharmaceutical acceptable salts, include, but are not limited to acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In some embodiments, the chemical structure of geranic acid, or 3,7-dimethyl-2,6-octadienoic acid, is:

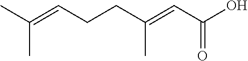

In some embodiments, geranic acid is in the form of a pharmaceutically acceptable salt. The type of pharmaceutical acceptable salts, include, but are not limited to salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Examples of acceptable organic bases include, but are not limited to, ethanolamine, diethanolamine, triethanolamine, tromethamine, and N-methylglucamine. Examples of acceptable inorganic bases include, but are not limited to, aluminum hydroxide, calcium hydroxide, potassium hydroxide, sodium carbonate, and sodium hydroxide.

In some embodiments, the choline and the fatty acid are synthesized using any suitable standard synthetic reactions. In some embodiments, the reactions are employed in a linear sequence to provide the compounds or they may be used to synthesize fragments which are subsequently joined by any suitable method. In some embodiments, the starting material used for the synthesis of choline or fatty acid is synthesized or are obtained from commercial sources.

In some embodiments, geranic acid is purified from the commercially available technical grade (Sigma-Aldrich, St. Louis, Mo.) by repeated (5-7×) recrystallization from a solution of 70 wt % geranic acid/30 wt % acetone at −70° C. In some embodiments, purity of the geranic acid is assessed by 1H NMR spectroscopy and conductivity measurements. In some embodiments, the term geranic acid refers to a geranic acid or a salt thereof. In some embodiments, the geranic acid is an anti-microbial agent.

In some embodiments, the pharmaceutically acceptable solvent is water, ethanol, diisopropyl adipate, polyethylene glycol (PEG), glycerin, propylene glycol, a short chain fatty acid, a fatty acid ester, or a combination thereof. In some embodiments, the pharmaceutically acceptable solvent is a liquid alcohol, liquid glycol, liquid polyalkalene glycol, liquid ester, liquid amine, liquid protein hydrolysate, liquid alkalated protein hydrolysate, liquid lanolin, lanolin derivative, or water. In some embodiments, the pharmaceutically acceptable solvent is diisopropyl adipate. In some embodiments, the composition is miscible with the pharmaceutically acceptable solvent. In some embodiments, at least one of the individual components of the composition is not miscible with pharmaceutically acceptable solvent. In some embodiments, the composition is miscible with diisopropyl adipate. In some embodiments, at least one of the individual components of the composition is not miscible with diisopropyul adipate. In some embodiments, the water is deionized water or Milli-Q® water. In some embodiments, the composition does not comprise a preservative. Examples of preservatives include, but are not limited to, a paraben or a phenoxyethanol.

The compositions described herein may be formulated in the form of a tablet, a capsule, a pill, a granule, a powder, an injection, a liquid, or a film. As used herein, “capsule” refers to a structure that encapsulates the ionic liquid compositions such that the ionic liquid composition is in the interior of the capsule. Capsule types include, but are not limited to polymers, oligosaccharides, and ethoxylates.

### II. THERAPEUTIC ANTIBODIES

Certain embodiments of the present invention is directed to an antibody that recognizes, binds, and/or neutralizes human leptin or a cell expressing the same. The antibody recognizes and specifically binds human leptin in its native form. Anti-leptin antibodies are commercially available and can be purchased from, for example, abcam, Waltham Mass. (Anti-Leptin antibody (ab16227)) and ThermoFisher Scientific (e.g., cat #PA1-051, PA1-052, 17436-1-AP, MA5-35984, MA5-35985). Leptin protein (GenBank NP-000221) can be purified or purchased (e.g., BioVision, Milpitas Calif.).

The term “antibody” is used herein in the broadest sense and refers generally to a molecule that contains at least one antigen binding site that immunospecifically binds to a particular antigen target of interest. The term “antibody” thus includes but is not limited to antibodies and variants thereof, fragments of antibodies and variants thereof, peptibodies and variants thereof, and antibody mimetics that mimic the structure and/or function of an antibody or a specified fragment or portion thereof, including single chain antibodies and fragments thereof. The term “antibody,” thus includes full-length antibodies or their variants as well as fragments thereof. Binding of an antibody to a target can cause a variety of effects, such as but not limited to, it modulates, decreases, increases, antagonizes, agonizes, mitigates, alleviates, blocks, inhibits, abrogates or interferes with at least one target activity or binding, or with receptor activity or binding, in vitro, in situ, and/or in vivo.

The term “monoclonal antibody” as used herein refers to an antibody obtained from a population of substantially homogeneous antibodies, i.e., the individual antibodies comprising the population are identical except for possible naturally occurring mutations that may be present in minor amounts. In certain aspects a monoclonal antibody that specifically binds an leptin peptide is described.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The terms “individual,” “subject,” and “patient,” used interchangeably herein, refer to an animal, preferably a mammalian (including non-primate and primate), including, but not limited to, murines, simians, humans, mammalian farm animals (e.g., bovine, porcine, ovine), mammalian sport animals (e.g., equine), and mammalian pets (e.g., canine and feline); preferably the term refers to humans.

As used herein, the terms “treatment”, “treating”, and the like, refer to obtaining a desired pharmacologic or physiologic effect. The effect may be therapeutic in terms of a partial or complete cure for a disease, symptom, and/or adverse effect attributable to the disease. “Treatment,” as used herein, includes administration of a compound of the present invention for treatment of a disease or condition in a mammal, particularly in a human, and includes: (a) inhibiting the disease, i.e., arresting its development; (b) providing palliative care, i.e., reducing and preventing the suffering of a patient; and (c) relieving the disease, i.e., causing regression of the disease or disorder or alleviating symptoms or complications thereof. Dosage regimens may be adjusted to provide the optimum desired response.

The anti-leptin antibodies may be monoclonal antibodies. Monoclonal antibodies may be prepared using hybridoma methods, such as those described by Kohler and Milstein (1975) Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The antibodies of the present invention may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

The anti-leptin monoclonal antibodies of the invention may be whole or an antigen-binding fragment of the antibody that binds to a leptin polypeptide or peptide, preferably a native sequence leptin polypeptide. Furthermore, in certain embodiments the monoclonal antibody is identified as having recognition of a leptin protein expressed by at least one cancer cell line or tumor tissue.

### III. METAL NANOPARTICLES (MNP)

As used herein, the term “metal nanoparticle” refers to a particle which is composed of metals and has a size of at least 1 nm to 100 nm. In particular, those prepared in conjunction with ionic liquids of the present invention are “metal nanoparticle composites”. Therefore, the metal nanoparticle composites may have anti-obesity activity as compared with metal nanoparticles prepared by other methods.

The metal nanoparticles can contain metal oxides selected from iron oxides (magnetite Fe₃O₄, maghemite γ-Fe₂O₃, or other Co ferrite or Ni ferrite), metals (Au, Ag, Cu, Si, Ge, Fe, Co etc), metal alloys (FeCo, FePt, CoPt, FeBi etc.) and metal chalcogenides (CdS, CdSe, CdSe, ZnS . . . etc.). The metal nanoparticles can have an overall size (average diameter) of approximately from 2, 10, 20, 30, 40, 50, 60, 70, 80, 90 to 100 nm including all values and ranges there between.

The metal nanoparticles contained in the ionic liquid compositions according to the invention may be mono-disperse (for example, they consist of a population having an average diameter centered around 10 nm), or mono- and/or poly-disperse (such as, for example, two populations having an average diameter centered around 4 nm and 10 nm), or else poly-disperse (different populations having different average diameters ranging from 2 nm and 20 nm).

The term “particle” refers to a small object, fragment, or piece of a substance that may be a single element, inorganic material, organic material, or mixture thereof. Examples of particles include, but are not limited to, polymeric particles, single-emulsion particles, double-emulsion particles, coacervates, liposomes, microparticles, nanoparticles, macroscopic particles, pellets, crystals, aggregates, composites, pulverized, and cross-linked protein or polysaccharide particles. In certain embodiments, the particle is a metal particle. A metal particle may be made of a single metal, or a mixture of metals (e.g., alloy). In certain embodiments, the metal particle comprises a transition metal. Examples of metal particles include, but are not limited to, gold, silver, copper, platinum, palladium, ruthenium, rhenium, iron, and nickel particles.

The term “nanoparticle” refers to a particle having an average (e.g., mean) dimension (e.g., diameter) of between about 1 nanometer (nm) and about 1 micrometer (am) (e.g., between about 1 nm and about 300 nm, between about 1 nm and about 100 nm, between about 1 nm and about 30 nm, between about 1 nm and about 10 nm, or between about 1 nm and about 3 nm), inclusive. In certain embodiments, the nanoparticle is less than 10 nm in diameter. In certain embodiments, the nanoparticle is about 3 nm in diameter.

### IV. KITS

Kits are provided which contain the necessary reagents to carry out the treatments or assays of the present invention. The kit may include one or more compartments, each to receive one or more containers such as: (a) a first container comprising one of the components of the present invention described above; and (b) one or more other containers comprising one or more of the following: wash reagents, reagents capable of detecting presence of the antibody or peptide.

The containers allow the efficient transfer of reagents from one compartment to another compartment such that the samples and reagents are not cross-contaminated, and the agents or solutions of each container can be added in a quantitative fashion from one compartment to another.

The kit typically contains containers that may be formed from a variety of materials, such as glass or plastic, and can include for example, bottles, vials, syringes, and test tubes. A label typically accompanies the kit, and includes any writing or recorded material, which may be in electronic or computer readable form (e.g., disk, optical disc, or tape) providing instructions or other information for used of the contents of the kit. The label indicates that the formulation is used for diagnosing or treating the disorder of choice.

One skilled in the art will readily recognize that the disclosed compositions of the present invention can be readily incorporated into one of the established kit formats that are well known in the art.

### V. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Ionic Liquid

Materials and Methods. Rat Animal Model. Rats are fed normal diet for 5 weeks, both male and female, body weight and food consumption is measured. Animals are fed a high fat diet for 5 weeks, body weight and food consumption is measured. Animals are fed high fat diet with CAGE-Leptin treatment for 5 weeks, body weight and food consumption is measured. Animals are fed high fat diet with BG-leptin treatment for 5 weeks, body weight and food consumption is measured.

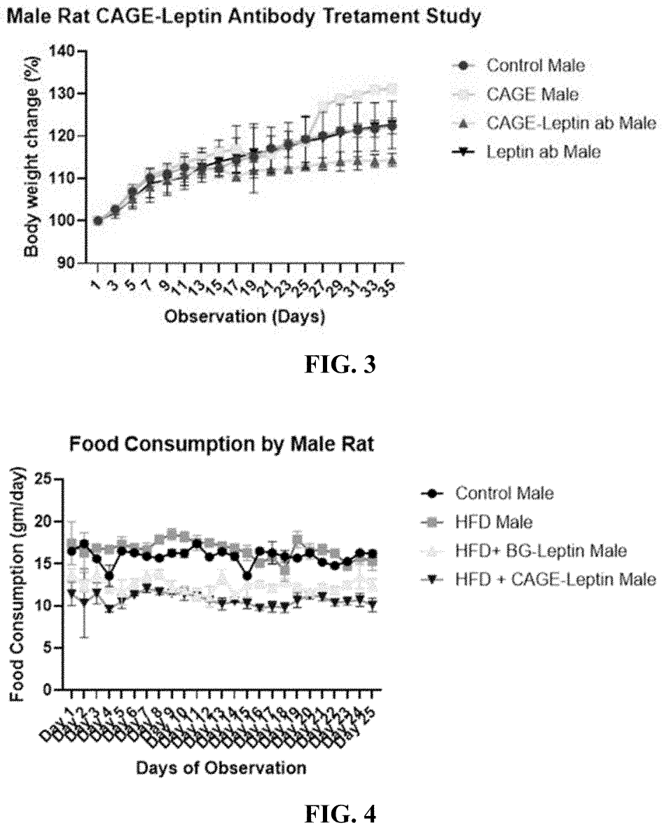

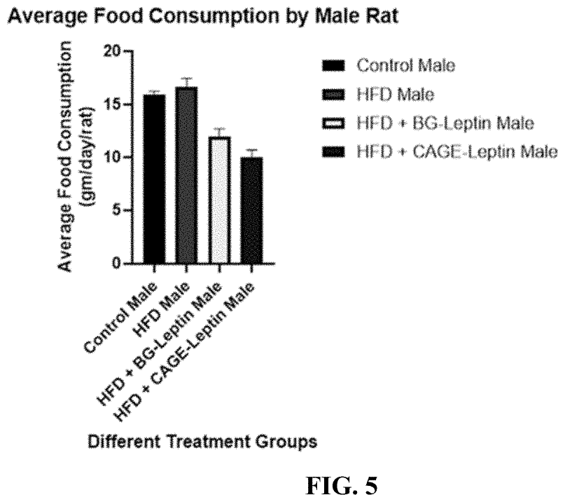

Male with leptin protein treatment gain body weight lower than regular and high fat diet male control group (FIG. 2). Four groups of male were getting high fat diet for 2 weeks and then we start administer with cage, cage-leptin ab and leptin ab treatments (FIG. 3). Leptin protein treatment of high fat diet group force the rat to consume lower amount of food compare to high fat and regular diet control group. In both graph we observe lower value of food consumption in BG-leptin and Cage-leptin treatment group (FIG. 4 and FIG. 5).

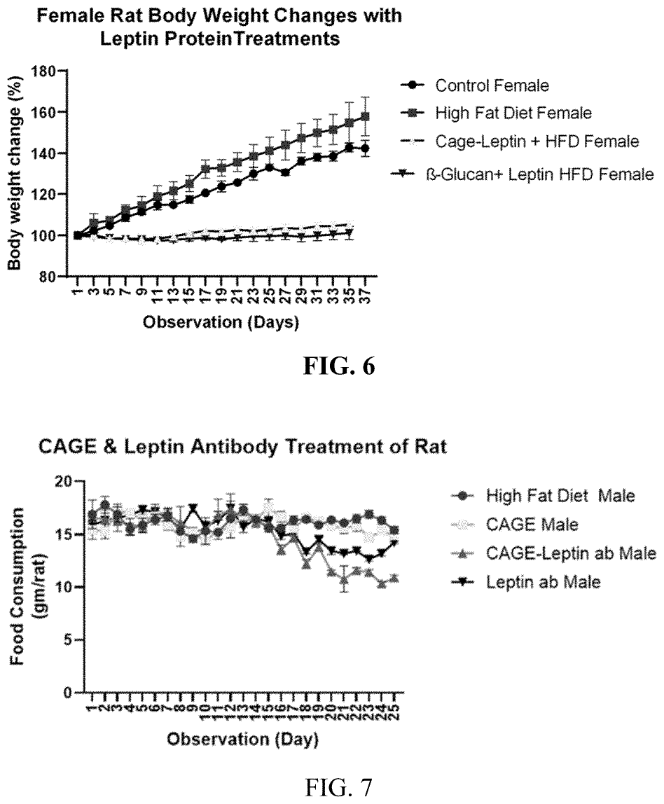

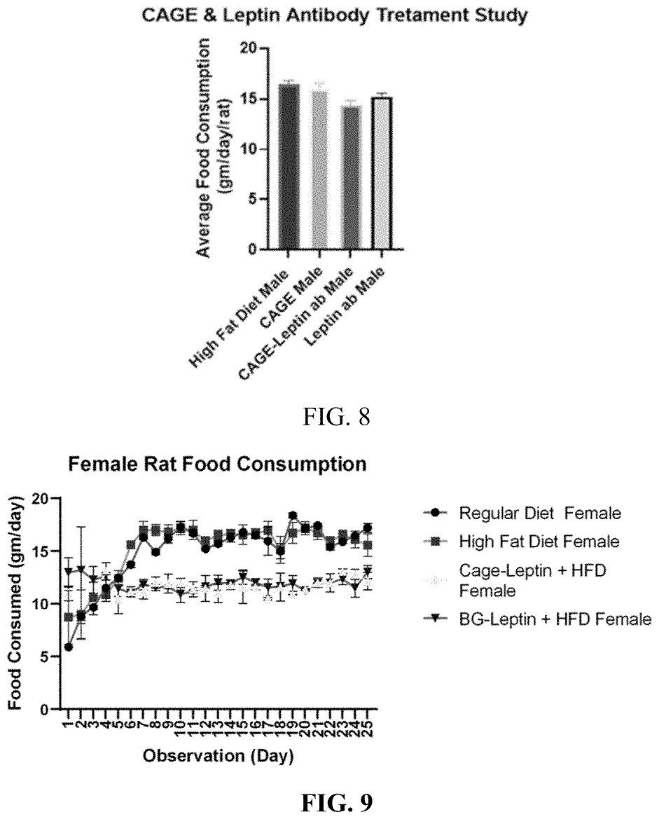

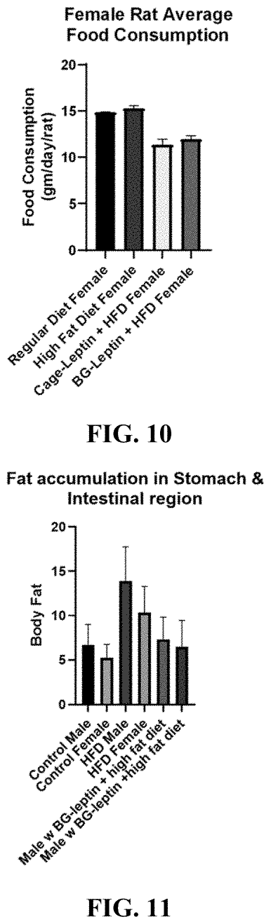

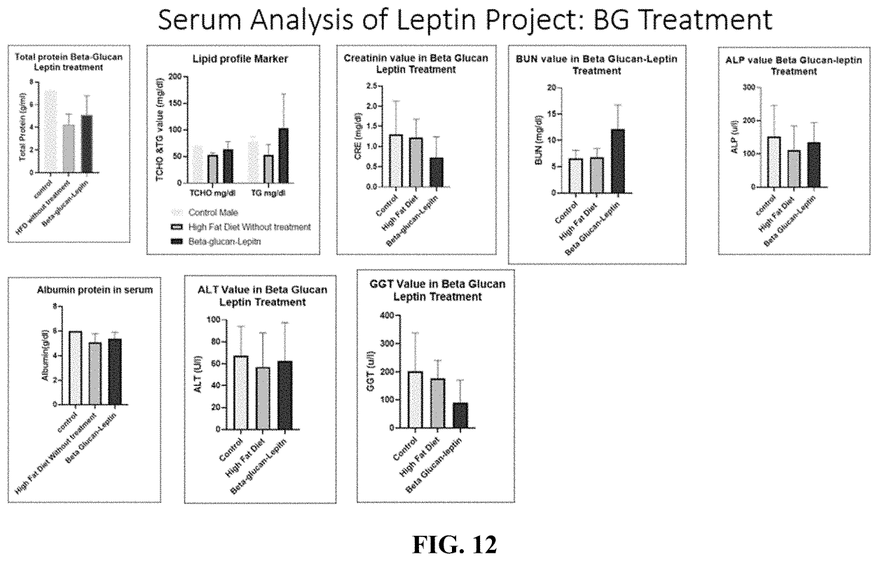

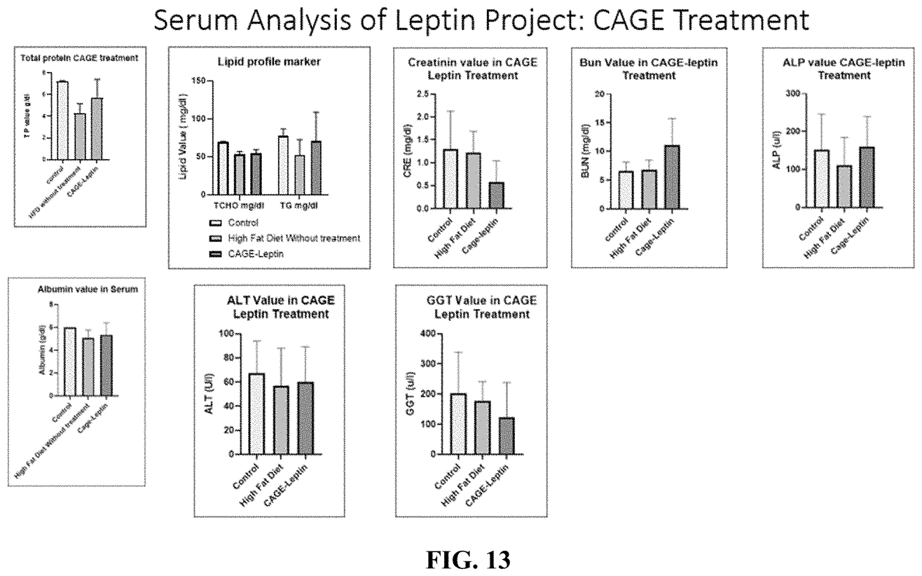

Treatment with leptin protein demonstrate despite consuming high fat diet female rat with CAGE-leptin and BG-leptin female group are not getting body weight compare to regular diet and high fat diet female groups (FIG. 6).

Tissue will be harvested, processed, and cut by cryosectioning. Serum and blood analysis of the samples will be completed. The tissue analysis like H&E staining, immunohistochemistry, Western blotting and mass spectrometry analysis will be performed. In vitro assays on CAGE-leptin and BG-leptin formulation stability and toxicity will be performed. The stability of leptin protein will be assessed by western blotting.

### Example 2—Metal Nanoparticle Incorporated Ionic Liquid

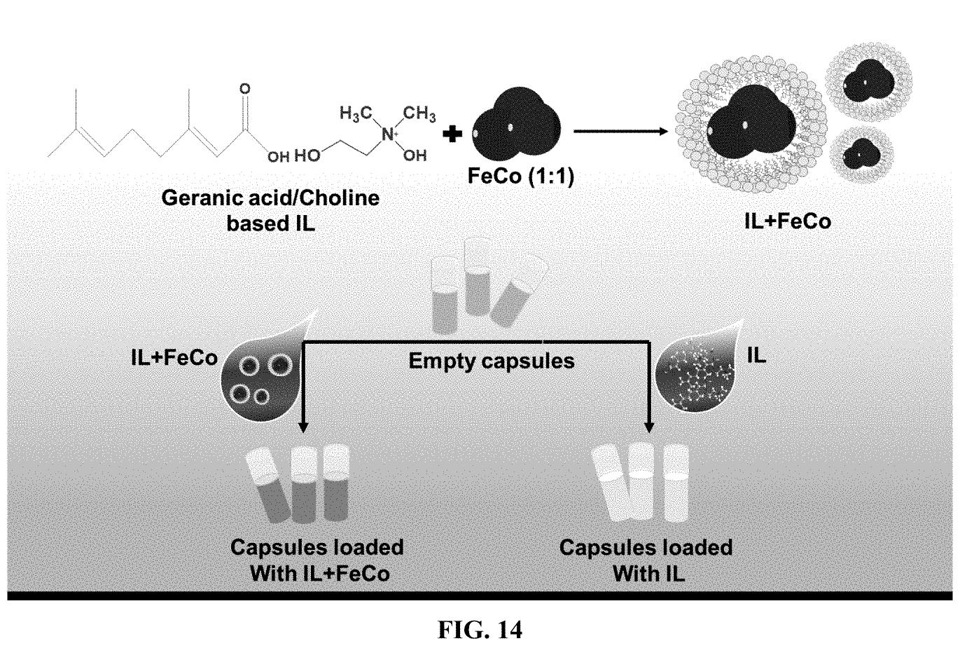

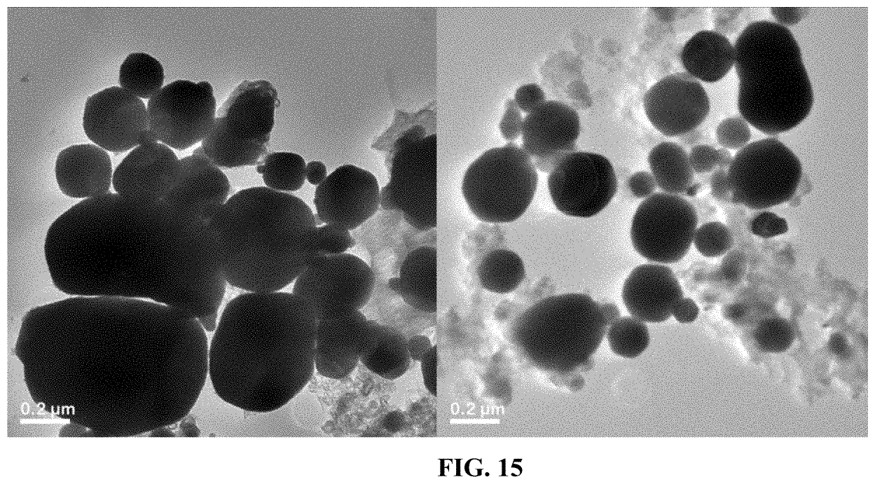

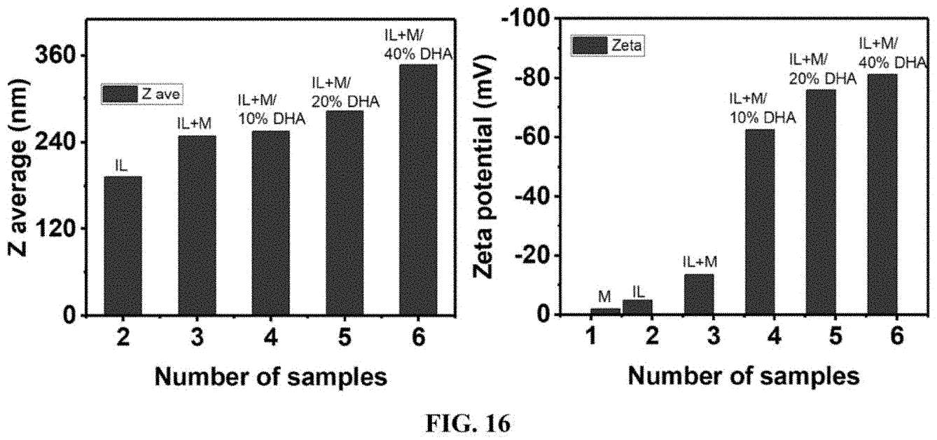

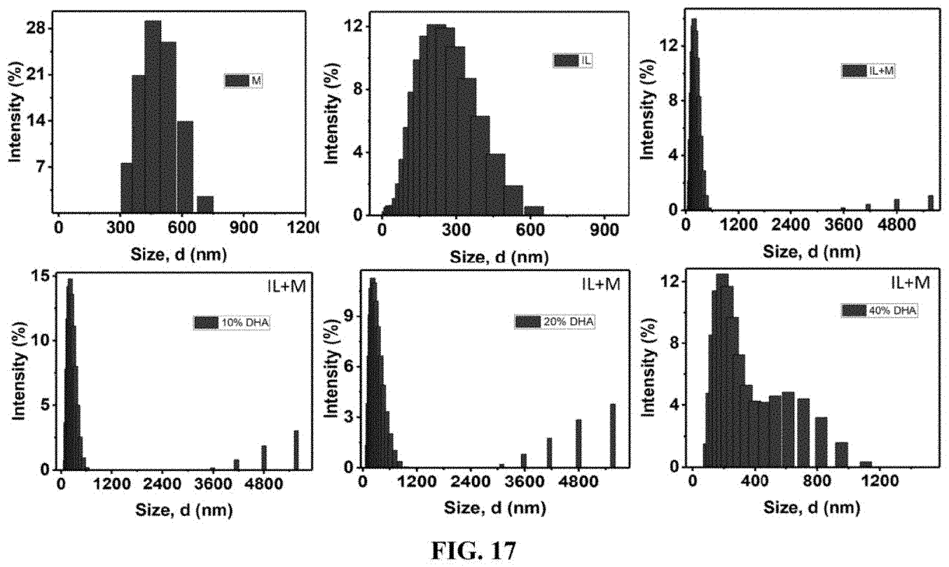

FIG. 14 is a schematic presentation of the preparation of IL/metal nanoparticles composite. FeCo bimetallic system was first prepared by following a two-step synthesis strategy including chemical mixing of Fe and Co salt with tissue paper (carbon source) followed by pyrolysis at 850° C. Approximately 4 mg metal NPs per 1 g of IL was mixed to prepare the composite. Then 20 mg of the formulation was loaded in the size 9 capsule. The capsules were named as IL and IL+M capsules. FIG. 15 to FIG. 17 provide characterization of the IL/MNP composites.

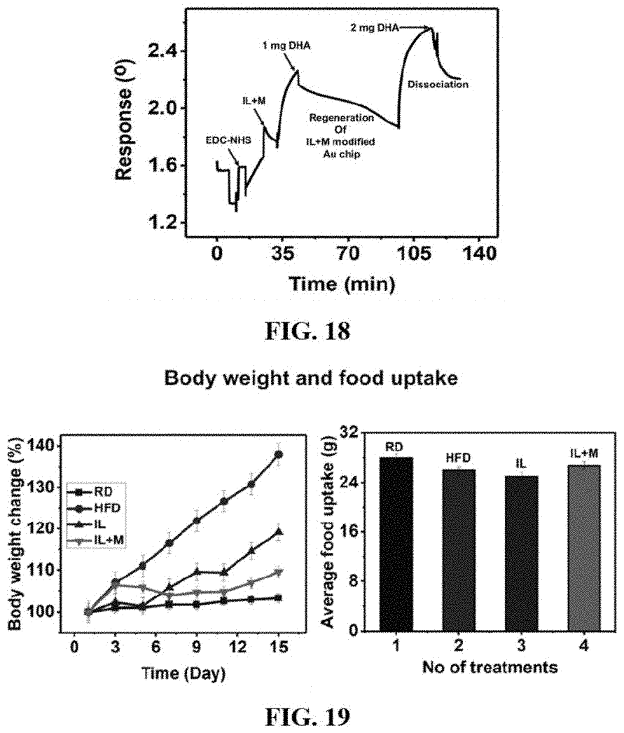

Surface Plasmon Resonance (SPR) Analysis of IL/MNP composites. FIG. 18 summarizes the studies of the binding affinity between IL+M and DHA by SPR was performed. Initially EDC-NHS was injected through the channel which showed slight increment of the sensogram response. Once IL+M composite dispersed in 1×PBS was passed, a sharp increment of the sensogram signal was observed which confirmed successful immobilization of IL+M on the COOH—Au chip. Finally, 1 mg of DHA was passed which interacted with IL+M profoundly and provided analyte signal. The regeneration buffer was used to regenerate the IL+M modified chip surface and finally a high concentration of DHA (2 mg) was passed which showed twice increment of the DHA signal indicating higher binding affinity of DHA with IL+M. RD=regular diet, HFD: High Fat Diet, IL+M=Ionic liquid+metal particle.

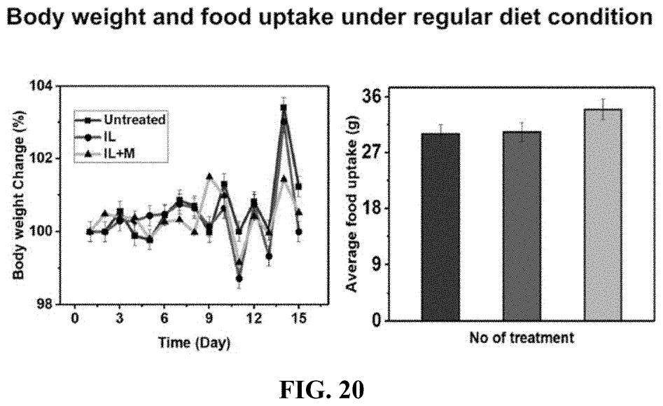

Body Weight and Food Uptake. FIG. 19 summarizes results from the study of the changes of the body weight and average food uptake of each of the groups of rats (Sprague-Dawley rats). Here, RD=Regular diet rats, HFD=High fat diet (60% extra fat) rats, IL=HFD rats having IL capsule, and IL+M=HDF rats having IL+M capsule. Each group contained 6 rats. IL and IL+M rats were given single pill orally everyday up to 15 days with HFD. While HFD rats were given only foods with 60% extra calories. Everyday the body weight and leftover food weight were measured. After 15 days of observations, the animals were sacrificed. The body weight of the IL+M group of rats reduced by almost 60% after 15 days of study compared to the HFD group of rats and the finding is way better than the IL group of rats. The food uptake by each of the groups was found consistent and similar indicating that the food uptake of the rats were not altered due to therapeutics intervention. RD=regular diet, HFD: High Fat Diet, IL+M=Ionic liquid+metal particle.

Body weight and food uptake under regular diet condition. FIG. 20 summarize results from the study of the effect of the therapeutics on regular diet conditions. In this study, three groups of rats (untreated, IL, and IL+M) were considered where all animals were given regular diet. The therapeutics showed no effects on body weight under regular diet condition which indicates that the therapeutics functioned only when fatty foods were given to the animals. This study suggests the mechanism of action of the therapeutics once given orally. The IL formulation can capture and bind to the lipid/fat molecules in the intestinal region where most of the absorption of nutrition occurs. This therapeutic interaction alters the process of further degradation and absorption of the nutrients from fats in the intestine. The reason of IL+M formulation exhibited better therapeutic efficacy is because the bimetallic nanoplatform provided a base for IL to capture more fats which was likely to increase the size of the particle upon capturing (also observed in size measurement, and SPR analysis). As a result, most of the nutrient absorption can be lowered and hence, the obesity condition can be improved. RD=regular diet.

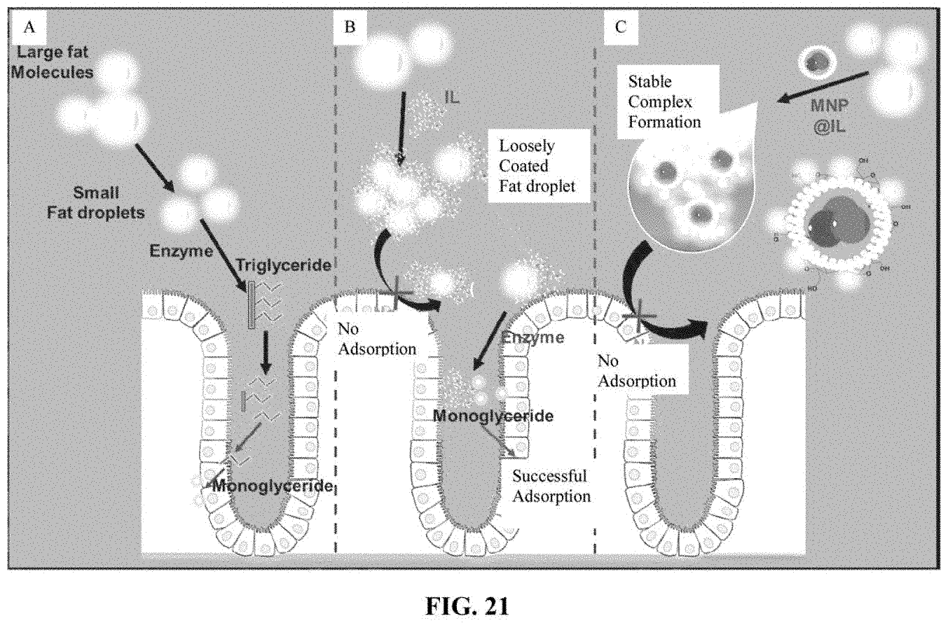

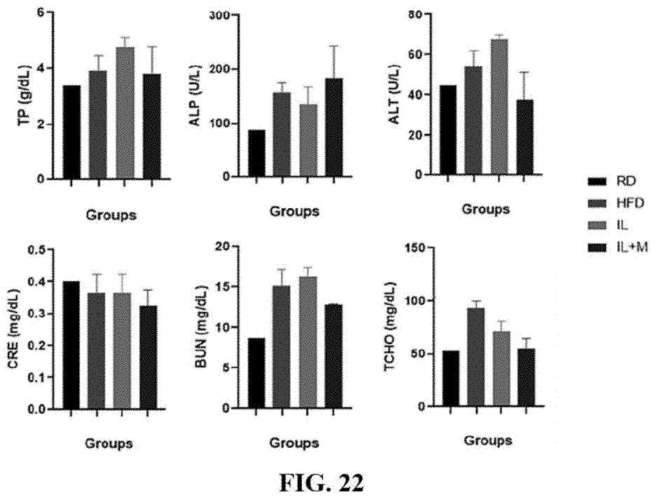

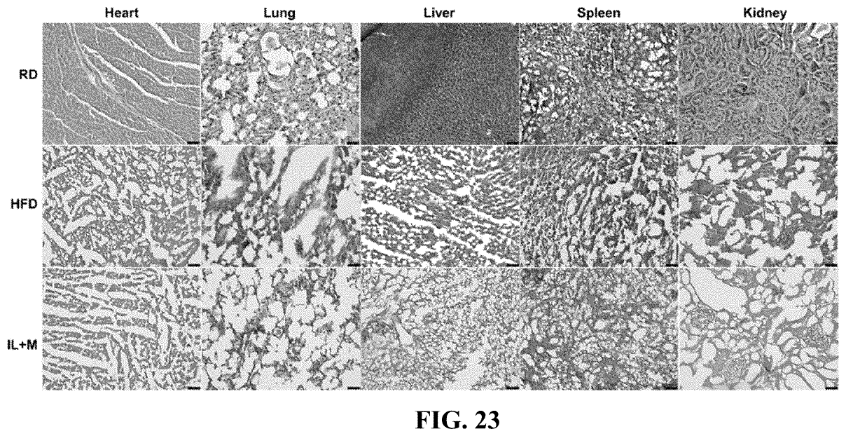

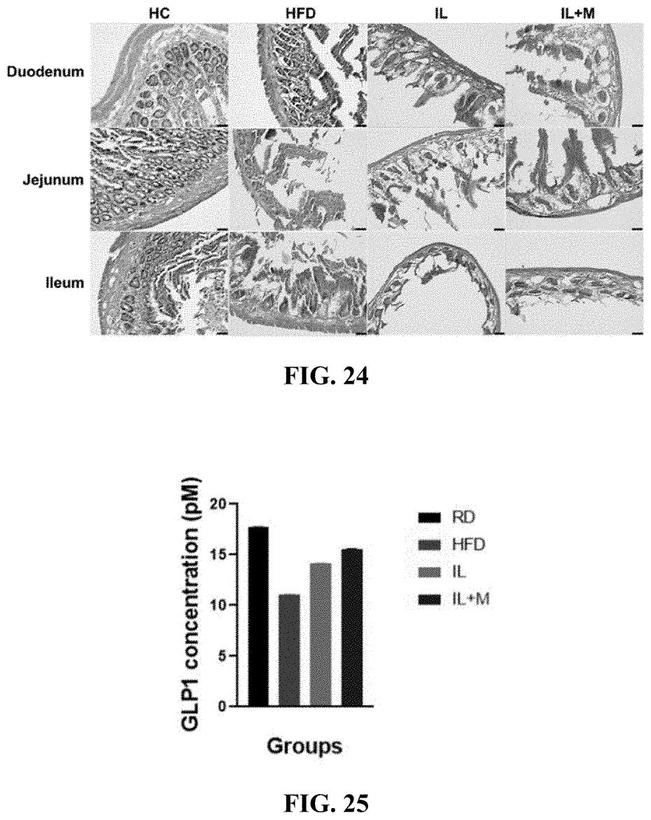

FIG. 21 illustrates of the mechanism of action related to IL and IL+MNP composites. The results and effect of IL and IL+MNP on serum biochemistry is presented in FIG. 22. Histology of major organs after 15 days of oral administration of IL and IL+MNP composites (FIG. 23) and of the small and large intestines after 15 days of administration of IL and IL+MNP composites (FIG. 24) demonstrate the effects on the composition at the tissue level. Serum GLP1 receptor levels were assessed after administration of IL or IL+MNP composites (FIG. 25).

1. An oral delivery composition comprising (i) a therapeutic agent and (ii) an ionic liquid or beta-glucan carrier.

2. The composition of claim 1, wherein the therapeutic agent is a protein or peptide.

3. The composition of claim 2, wherein the protein is an antibody.

4. The composition of claim 3, wherein the antibody is an anti-leptin antibody.

5. The composition of claim 2, wherein the peptide is leptin.

6. The composition of claim 1, wherein the ionic liquid is a choline:geranic acid ionic liquid.

7. The composition of claim 6, wherein the choline:gernate is at a ratio of 1:1, 1:2, 1:3, or 1:4 of choline to geranic acid.

8. The composition of claim 1, wherein the therapeutic agent is or further includes DHA or EPA.

9. The composition of claim 1, wherein the therapeutic agent is or further includes metal nanoparticle (MNPs).

10. The composition of claim 9, wherein the MNP is an iron-cobalt (FeCo) MNP.

11. A method for oral delivery of an antibody comprising orally administering an effective amount of a composition of claim 2 to a subject in need of an antibody therapy or administration.

12. The method of claim 11, wherein the antibody is an anti-leptin antibody.

13. The method of claim 11, wherein the subject is in need of weight loss.

14. The method of claim 11, wherein the antibody is provided at a dose of 5 to 10 μg.

15. The method of claim 11, further comprising administering a hormone based weight loss therapy.
